(19) United States
(12) Patent Application Publication
(10) Pub. No.: US 2023/0012664 A1
(43) Pub. Date: Jan. 19, 2023

(54) SELECTIVE DRUG DELIVERY IN AN ION PUMP THROUGH PROTON ENTRAPMENT

(71) Applicant: OBOE IPR AB, Linköping (SE)

(72) Inventors: Xenofon STRAKOSAS, Norrköping (SE); Daniel T. SIMON, Linköping (SE); Maria SEITANIDOU, Norrköping (SE); Magnus BERGGREN, Norrköping (SE)

(21) Appl. No.: 17/786,883

(22) PCT Filed: Dec. 18, 2020
(86) PCT No.: PCT/EP2020/087294

§ 371 Date: Jun. 17, 2022

(30) Foreign Application Priority Data
Dec. 20, 2019 (SE) 1951545-1

(51) Int. Cl. A61N 1/04

(52) U.S. Cl. CPC A61N 1/0436; A61N 1/0448; A61N 1/0444; A61N 1/303

(57) ABSTRACT

A device (100) for electrophoretic delivery of ions comprising a source electrode (200) in electric and ionic contact with a source electrolyte (202), and a target electrode (400) in electric and ionic contact target electrolyte (402), said source and target electrodes (200, 400) capable of conducting ions and electrons; an ion-conductive channel (302) connecting the source electrolyte (202) with the target electrolyte (402) to provide an ionic connection between said source and said target electrodes (200, 400), wherein said electrodes (200, 400) and said ion-conductive channel (302) are formed of solid or semi-solid materials, and a controller, operable to apply a drive voltage between said source and said target electrodes (200, 400), such that at least after a voltage is applied across said ion-conductive channel, a potential difference between said source and target electrodes (200, 400) is provided, further comprising a trapping electrode (300) comprising an effective amount of a Bronsted base, said trapping electrode (300) being arranged in ionic contact with the ion-conductive channel (302). Use of the device is also disclosed, as is a method of operating the device and a method electrophoretic delivery of ions.

### TECHNICAL FIELD

The present disclosure relates to the field of organic bioelectronics, more specifically a device for electrophoretic delivery of ions from a source to a target electrolyte, and to an apparatus for transporting ions to or from a cell.

In particular, the present disclosure relates to a more efficient delivery of cationic species of drugs enabled by the selective entrapment of protons from the drug containing electrolyte. The present disclosure further relates to use of such a device and device system, as well as to a method of operating such a device.

### BACKGROUND

Drugs typically have their therapeutic action at specific sites in the body but are often administered systemically. This means that only a small portion of the drug ends up where it is needed, and the rest may cause side effects elsewhere in the body. By delivering drugs locally, where and when they are needed, a much lower dose can be used, and hence side effects may be avoided. Indeed, many drugs that today fail in clinical trials because of their adverse effects due to high dosages could in fact be effective and without side effects if they were delivered locally and at very low doses.

Neurological disorders, such as Parkinson's disease and epilepsy result from perturbations to the nervous system. Treatments for neurological disorders include administration of pharmaceuticals and electrical stimulation of the nerves. With electrical stimulation a very fast effect is achieved, but the effect cannot target a certain nerve signalling pathway but rather effects all nearby nerve cells. Pharmaceutical treatment, on the other hand, is more specific, but much slower since the drugs need to travel to the site of action.

A drug delivery device, such as an implantable device that could release drugs locally, and on a minute-to-second (or faster) timescale would have the benefits of specificity that pharmaceutical treatment has, along with the faster response typical of electrical stimulation. Also, the side effects due to high dosage that is associated with systemic administration of drugs, as well as side effects associated with electrical stimulation, such as muscle twitching and sensation, could be avoided.

There are several methods for local drug delivery already in use, including implanted pumps where the delivery rate can be controlled in time. When the drug is dissolved and delivered in a carrier fluid, the environment where the drug is delivered is diluted. This can further lead to an increased pressure if the drug is delivered into a confined compartment. Microfluidics is the scaled down version of drug delivery in fluids, mostly used for in vitro lab-on-a-chip applications. Even though the volumes are much smaller, the same problem with increased pressure still exists. Furthermore, the amount of delivered drug is not controlled to a very high extent for either of these fluidic techniques. Other techniques used in practice are transdermal patches and subdermal implants that exhibit passive delivery, meaning that drugs are continuously released at a predetermined rate. The delivery rate can thus not be actively controlled in time with a sufficiently high degree of precision.

A few techniques for local drug delivery utilize the fact that many drugs and neurotransmitters are, or can occur in, electrically charged form. This implies that they can be controlled and measured electrically. These techniques include drug release from conducting polymers and iontophoresis. Iontophoresis, or electromotive drug administration (EMDA), is a method for administering charged drugs with an applied electric field directly into the target, e.g. tissue or cell clusters. This method is not very exact in terms of the amount of delivered drugs.

Charged drugs have also been incorporated as counter ions into conducting polymers, and when the charge of the polymer is altered as a function of oxidation or reduction, the drug (acting as counter ion to the charged polymers) is expelled and released from the conducting polymer without any liquid flow (as shown in FIG. 17). Although many research groups have successfully used this principle, it suffers from high passive leakage, since ions of the electrolyte/body fluid are passively exchanged with the ionic drugs loaded in the conducting polymer, regardless of the addressing voltage. Furthermore, only the drugs originally incorporated into the conducting polymer during the fabrication or pre-usage phase can be released, which limits the amount of drug that can be delivered.

An Organic Electronic Ion Pump (OEIP) is an alternative solution to deliver charged drugs/biomolecules from micrometer-sized outlets without any significant fluid flow. The delivery rate of drugs from an OEIP is actively controlled by the applied current and can thus be tuned.

WO 2017/157729 A1 discloses such a conductive drug delivery device with controlled delivery electrode. However, during pumping of cations, protons (H⁺), which exhibit high mobilities, are also delivered which can induce side effects, such as altering pH and disrupting normal function. In certain circumstances, e.g. specific pH or pKₐ of drug, the amount of protons delivered could even exceed the amount of drug delivered.

A method of removing protons from the electrolyte is disclosed in EP 0528789 A1, where chelating agents are facilitated to bind the protons.

However, this process is non-reversible and cannot easily be adjusted to a specific pH.

Another method for the removal of protons is described in WO 9617649 A1. There both protons and hydroxides (OH⁻) are trapped by a polymer. Also, this method is non-reversible and further has the problem of saturating the polymer with ions after extensive use.

Thus, there is a need for a device, which may be an implantable device, that can deliver drugs with an actively controlled amount without also transporting protons to the target environment as it can lead to unwanted side effects. Further, drug delivery should be fast and trapping of protons should not affect the pH in the drug reservoir.

### SUMMARY

It is an object of the present disclosure, to provide an improved OEIP for selective delivery of cationic species, which eliminates at least some of the disadvantages of the prior art devices.

The invention is defined by the appended independent claims. Embodiments are set forth in the appended dependent claims and in the following description and drawings.

According to a first aspect, there is provided a device for electro-phoretic delivery of ions comprising a source electrode in electric and ionic contact with a source electrolyte, and a target electrode in electric and ionic contact with a target electrolyte, said source and target electrodes capable of conducting ions and electrons; an ion-conductive channel connecting the source electrolyte with the target electrolyte to provide an ionic connection between said source and said target electrodes, wherein said electrodes and said ion-conductive channel are formed of solid or semi-solid materials, and a controller, operable to apply a drive voltage between said source and said target electrodes, such that at least after a voltage is applied across said ion-conductive channel, a potential difference between said source and target electrodes is provided, wherein the device further comprises a trapping electrode comprising an effective amount of a Bro/nsted base, said trapping electrode being arranged in ionic contact with the ion-conductive channel.

The device is thus based on the previously disclosed OEIP, however the device of this disclosure comprises further at least one trapping electrode. The addition of a trapping electrode in the device allows the selective trapping of protons within the ion-conductive channel, which is connecting the source with the target electrolyte.

Therefore, the device is able to deliver drugs of interest to a target solution without liquid flow into the target as previous disclosed OEIPs but additionally provides a higher efficiency of ions delivered, using the ratio of electrons recorded in the driving circuit to the drug molecules delivered to target, a reduced influence on the pH value in the target solution due to concomitant proton delivery, a faster delivery of a known low concentration of drug, and the possibility to correlate the current recorded and the measured concentration of the ion to be delivered.

The term electrophoretic is a general term that describes the migration and separation of ions under the influence of an electric field which is used to drive the controlled delivery of the ions.

Ions being defined as an atomic or molecular particle having a net electric charge. (IUPAC. Compendium of Chemical Terminology, 2nd ed. (the “Gold Book”). Compiled by A. D. McNaught and A. Wilkinson. Blackwell Scientific Publications, Oxford (1997))

Hence, the device is intended to transport charged and biologically active molecules, macromolecules such as amino acids, vitamins, peptides, neurotransmitters, hormones, and other substances, e.g. pharmaceuticals, endogenous substances or charged metal ions. Further, the device is particularly intended to deliver cationic species of said ions. Examples of ions to be transported are small drug molecules that are zwitterions, and which also need to be protonated to be delivered, e.g. γ-aminobutyric acid (GABA) and glutamate.

The effect of trapping protons with an additional trapping electrode is based on the proton accepting property of Bro/nsted bases, which in this disclosure, refers especially to proton acceptors that specifically accept protons if a negative potential is applied, and hence negatively biasing the trapping electrode. The trapping electrode may be configured for selectively trapping protons when a negative bias is applied to the trapping electrode with respect to the electrolyte. The term negatively biased refers to the trapping electrode being relative to the other electrodes more negatively biased. Hence, the trapping electrode may be the lowest voltage point. Different elements and molecules can act as a Bro/nsted base, e.g. late transition metals, metal hydrides, electrically conductive polymers and combinations thereof.

To achieve the proton trapping effect a minimum active amount of the Bro/nsted base is present in or at the trapping electrode. The minimum active amount is dependent on the ion to be delivered, the pH, and the Bronsted base used. In the case of GABA, as ion to be transported, and palladium (Pd), as active Bronsted base, optimal minimum concentration of Pd is twice the concentration of GABA.

According to tests a device based on Pd as active Bronsted base in the trapping electrode improved the drug delivery rate by two orders of magnitude compared to an ion-pump without the trapping electrode but with the same specific geometry. Further, the device based on Pd as active Bronsted base in the trapping electrode traps the proton reliably so that no significant change of pH value occurs in the target electrolyte due to the ion delivery.

The high trapping efficiency and reliability allows further to correlate the current delivered to the target with the concentration of delivered drug ions in the target electrolyte, which was not possible with traditional ion-pump. Thereby more control over the ions delivered can be achieved.

The trapping electrode may act as a Bronsted acid after it was first loaded with protons and it is in a neural or positive potential versus the electrolyte potential.

The trapping electrode may be arranged in electric contact with at least one of the source and target electrodes. The electronic contact may be operated in a way that the trapping electrode is ground.

The ion-conductive channel may at least be partially formed of a cation exchange membrane (CEM).

The source electrolyte may comprise a neurotransmitter, e.g. GABA and/or glutamate in a form suitable for transport by said cation exchange membrane.

The controller may be operable to apply a negative bias with respect to the other electrodes to the trapping electrode. Negatively biased refers to the trapping electrode being relative to the other electrodes more negatively biased. Hence, the lowest voltage point is at the trapping electrode.

The Bronsted base may comprise at least one of the materials selected from the group consisting of late transition metals, metal hydrides, electrically conductive polymers and/or combinations thereof.

The trapping electrode may comprise at least an effective amount of the materials selected from the group consisting of late transition metals, metal hydrides, electrically conductive polymers and combinations thereof.

The device may further comprise a source electrolyte retainer, for retaining the source electrolyte in contact with the source electrode, and/or a target electrolyte retainer, for retaining the target electrolyte in contact with the target electrode.

The trapping electrode may be positioned closer to the target electrolyte retainer than to the source electrolyte retainer.

Further, the device further may comprise at least one second trapping electrode in ionic contact with the ion-conductive channel.

The source electrolyte may have a pH value of 7 or lower.

The device may comprise at least one waste channel, each waste channel comprising a waste electrolyte and a waste electrode.

The device may comprise at least two source electrolytes. The source electrolytes may comprise the same or different ions for electrophoretic delivery.

The device may comprise at least two ion-conductive channels, wherein the controller is operable to apply different potentials to the different trapping electrodes to selectively transport ions through a at least one of the ion-conductive channels.

According to a second aspect, there is provided a device for medical use according to the first aspect, for electrophoretic delivery of ions in conjunction with the entrapment of protons to a target electrolyte. The target electrolyte may comprise any one of tissue, body fluids, cells, cell medium, physiological fluids, and biological environments.

According to a third aspect, there is provided a method of electrically controlled transport of ions between a source electrolyte and a target electrolyte by a device according to the first aspect, comprising the steps of applying a first potential to the source electrode, applying a second potential to the target electrode, said second potential being lower than the first potential, whereby cations are driven to migrate through the ion-conductive channel from the source electrolyte to the target electrolyte, and applying a third potential to the trapping electrode, said third potential being lower than the second potential, whereby protons present in the ion-conductive channel are attracted to the trapping electrode.

The method may further comprise the additional steps of recording a current applied over time to the target electrode, and calculating a concentration of ions/drug/biomolecules delivered to the target electrolyte based on said recorded current; switching of the third potential, and releasing the proton trapped at the trapping electrode and thereby recovering the trapping electrode.

According to a fourth aspect, there is provided a method of electrically controlled transport of ions between a source electrolyte and a target electrolyte, comprising the steps of providing said ions in the source electrolyte, providing an ion-conductive channel comprising a solid or semi-solid material, for ionically connecting the source electrolyte with the target electrolyte, applying a first potential difference to a source electrode contacting the source electrolyte and a second potential to a target electrode contacting the target electrolyte, a difference between said first and second potentials being sufficient to drive said ions through the ion-conductive channel from the source electrolyte to the target electrolyte, applying a third potential to a trapping electrode comprising an active amount of a Bronsted base, wherein said third potential is sufficiently low so as to attract protons present in the ion-conductive channel, such that said protons are prevented from reaching the target electrolyte.

The method may further comprise the steps of applying a potential, wherein the first potential is greater than the second potential, and the second potential is greater than the third potential, and first and second potentials are positive, and the third potential is negative.

The method may further comprise the additional steps of recording a current applied to the target electrolyte as a function of time and calculating an amount of ions delivered to the target electrolyte based on said recorded current; releasing the third potential, whereby protons trapped at the trapping electrode are released into the ion-conductive channel; applying a fourth potential to the waste electrode to fill the waste channel with ions from the ion-conductive channel.

### BRIEF DESCRIPTION OF THE DRAWINGS

FIG. 1 is a schematic side view of the device comprising two electrodes, an ion-conductive channel and a trapping electrode.

FIG. 2 is a schematic side view of the device comprising two electrodes, an ion-conductive channel, a trapping electrode and a waste electrode.

FIG. 3 is a schematic side view of the device comprising two electrodes, an ion-conductive channel, a trapping electrode, and at least a second trapping electrode.

FIG. 4 is a schematic side view of the device comprising two electrodes, two ion-conductive channels with each comprising a trapping electrode.

FIG. 5 is a schematic top view of the device comprising two electrodes, two ion-conductive channels with each comprising a trapping electrode.

### DETAILED DESCRIPTION

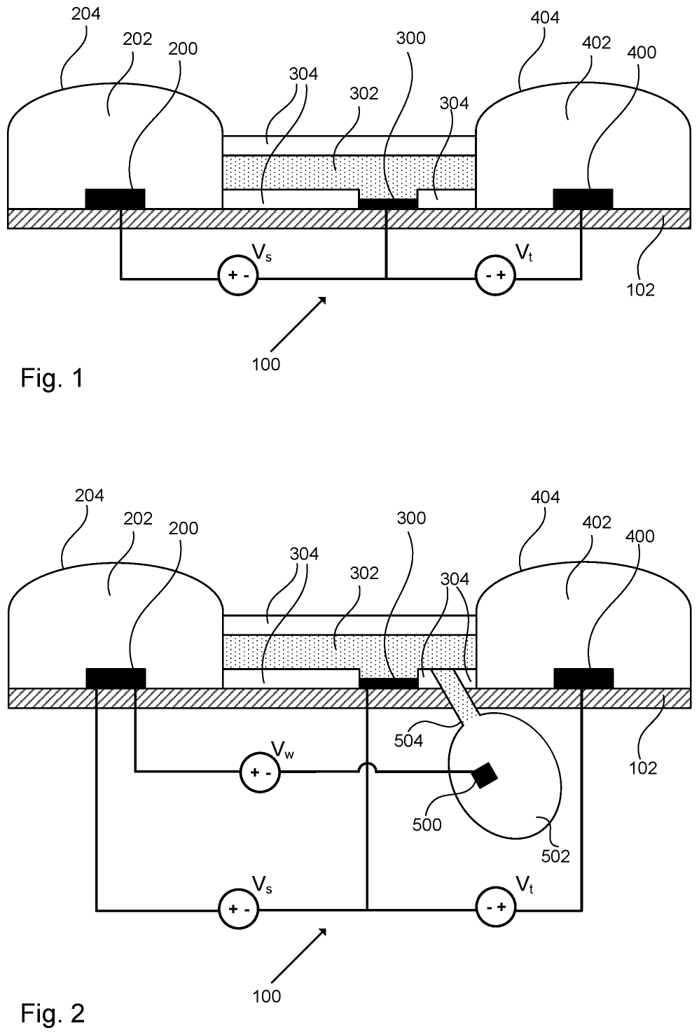

FIG. 1 illustrates a device 100 according to present disclosure describing a source electrolyte 202 and a target electrolyte 402. The electrolytes are ionically connected by an ion-conductive channel 302.

The electrolyte for use with the device or method may be based on a solvent that permits ionic conduction in the electrolyte, i.e. that allows for the dissociation of ionic substances such as salts, acids, bases, etc. The solvent and/or the ionic substance may contribute nucleophiles. Possible electrolytes for use in combination with the device are solutions of salts, acids, bases, or other ion-releasing agents in solvents that support the dissociation of ionic species, thus allowing ionic conductivity. In applications where it is required, the target electrolyte may comprise buffer solutions, such as buffer solutions suitable for use with living organisms or biomolecules, such as proteins. Examples of such buffers include NaHPO₄ and sodium acetate. As other non-limiting examples of possible electrolytes, mention can be made of: aqueous solutions of potassium acetate, calcium acetate, NaCl, Na₂SO₄, H₃PO₄, H₂SO₄, KCl, RbNO₃, NH₄OH, CsOH, NaOH, KOH, H₂O₂; organic solvents such as acetonitrile, pyridine, DMSO, DMF, dichloromethane, etc., in combination with suitable salts, such as lithium perchlorate and tertiary ammonium salts, e.g. tetra-butyl ammonium chloride; inorganic solvents such as hypercritical CO₂, liquid SO₂, liquid NH₃, etc., in combination with salts that dissociate in these solvents; solvents displaying auto-dissociation, which results in the formation of ionic species, such as water, formic acid and acetic acid.

The term electrolyte also encompasses solutions comprising charged biologically active molecules or macromolecules such as charged amino acids, proteins, vitamins, peptides or hormones. An electrolyte may also comprise cell culturing media or ingredients thereof, such as proteins, amino acids, vitamins and growth factors.

Many drugs and neurotransmitters present higher transport efficiency at low source pH. At pH 3, GABA neurotransmitter structure leads to a more globular conformation and as consequence higher mobility of the cationic GABA through the channel. This phenomenon results in the problem stated above since significant shifts in pH lead to an abundance of protons, which are delivered along with the neurotransmitter affecting the channel selectivity and it can create side effects for implantable devices. In one embodiment the source electrolyte 202 has a pH value of preferably 3-5.

The electrolyte may also be in a semi-solid or solidified form, for example comprising an aqueous or organic solvent-containing gel as described above. However, solid polymeric electrolytes are also contemplated. Furthermore, the term electrolytes also encompass liquid electrolyte solutions soaked into, or in any other way hosted by, an appropriate matrix material, such as a paper, a fabric or a porous polymer.

The source electrolyte 202 contains the ions to be delivered. The target electrolyte 402 may be the body fluid or the cell culture medium, or whatever media needed for the application. Thus, the content of this electrolyte 402 can often not be controlled, and it may be determined from the application of the device.

A first and a second material, as e.g. in this embodiment the source and the target electrolyte, have an ionic connection or are in ionic contact when a substantial number of ions comprised in the first material can move from the first material to the second material, possibly via a third material. The ionic movement may be caused by diffusion or by an applied electric field.

A material which provides an ionic connection between a first and a second material, is a material which is ionically conductive, and in ionic contact with both said first and said second material.

The ion-conductive channel 302 may be a charge selective membrane such as a cation or anion exchange membrane (CEM or AEM). In the present disclosure the charge selective membrane is a CEM to selectively allow the delivery of cationic species. CEMs are characterized by a high concentration of fixed negative charges and the permselectivity holds if the ionic concentrations in the adjacent electrolytes are lower than the fixed charge concentration of the CEM (Donnan exclusion). The ionic current through the membrane is represented by the combination of migration controlled by the electric field and diffusion along concentration gradients, with diffusion most noticeable when no potential is applied. CEMs only allow transport of cationic species, e.g. cations of atoms, biologically active molecules, and macromolecules from the source and to the target electrolyte. The CEM should further be adapted in such a way that is allows for the transportation of the desired ion, which means that ions larger than protons need to be able to pass through but that a certain size selectivity is upheld to not disrupt the charge selectivity. A CEM material can be a polymeric membranes with fixed negative charges that are ionically active, i.e. overoxidized PEDOT:PSS, PVA:PSS, Nafion (diluted), polyacrylic acid(PAA), and PEG:PAA. An example of a CEM is polyanion poly(4-styrenesulfonic acid-co-maleic acid (PSS-co-MA) cross-linked with the polyalcohol polyethylene glycol (PEG), where PSS is the primary ion exchange group, with a final SU-8 photoresist layer as encapsulation layer 304. The encapsulation layer 304 may be made of other solid of semi-solid materials. The ion-conductive channel 302 may be directly or indirectly attached to the device.

The term semi-solid material refers to a material, which at the temperatures at which it is used has a rigidity and viscosity intermediate between a solid and a liquid. Thus, the material is sufficiently rigid such that it does not flow or leak. Further, particles/flakes in the bulk thereof are substantially immobilized by the high viscosity/rigidity of the material.

For example, a semi-solid material may have the proper rheological properties to allow for the ready application of it on a support as an integral sheet or in a pattern, for example by conventional printing methods. After deposition, the formulation of the material may solidify upon evaporation of solvent or because of a chemical cross-linking reaction, brought about by additional chemical reagents or by physical effect, such as irradiation by ultraviolet, infrared or microwave radiation, cooling etc.

The semi-solid or solidified material may comprise an aqueous or organic solvent-containing gel, such as gelatine or a polymeric gel.

The drawing of the device 100 in FIG. 1 shows further a source electrode 200 and a target electrode 400. The source electrode 200 is arranged in electrical and ionic contact with the source electrolyte 202. The target electrode 400 is arranged in electrical and ionic contact with the target electrolyte 402. The source and target electrodes 200, 400 that control the potential of the respective electrolytes 202, 402 may each comprise a material or a combination of materials which is capable of electron-to-ion conversion, i.e. they need to enable charge transfer between the electrode and its contact and the electrodes may be so called non-polarizable electrodes. The materials used as source and target electrodes 200, 400 are electrochemically active. The source and the target electrodes 200, 400 may be made of Au, Ag/AgCl or a conducting polymer such as PEDOT. The source and the target electrodes 200, 400 may be directly or indirectly attached to the device.

The device 100 may comprise a source electrolyte retainer 204 for retaining the source electrolyte 202 in contact with the source electrode 200 and/or a target electrolyte retainer 404 for retaining the target electrolyte 402 in contact with the target electrode 400. The target electrolyte 402 does not need to be restricted to a simple retainer 404 as it can be allowed to spread within the desired target, e.g. the target tissue. The source electrolyte retainer 204 and the target electrolyte retainer 404 may be directly or indirectly attached to a support 102 or its walls may form partially part of the support 102 and its layers. The source electrolyte retainer 204 and the target electrolyte retainer 404 may be in the form of a recess within the support 102 and its layers, or they may be in the shape of a separate vessel formed of solid or semi-solid material. The source electrolyte retainer 204 and the target electrolyte retainer 404 may be made of a hydrophobic confinement within a SU-8 patter. The support 102 may be glass substrate or a coated support. The coat may be a metal layer, e.g. Cr or Au, or photoresist such as SU-8 or a combination of different layers.

Two parts which are directly attached to each other are in direct physical contact with each other. When a first part is directly attached to a second part, which second part is directly attached to a third part, said first and third parts are referred to as being indirectly attached to each other. Similarly, when said third part is directly attached to a fourth part, said first and fourth parts are referred to as being indirectly attached to each other.

The device 100 in FIG. 1 shows further a trapping electrode 300 which may comprise a material or a combination of materials which is capable of electron-to-ion conversion, i.e. they need to enable charge transfer between the electrode and its contact and the electrodes may be so called non-polarizable electrodes. The materials used as trapping electrodes are electrochemically active. The trapping electrode comprises an active amount of a Bronsted base. Different elements and molecules can act as a Bronsted base, e.g. late transition metals, metal hydrides, electrically conductive polymers and combinations thereof.

The term late transition metals refers to d-block elements from group 8 to 11, e.g. Fe, Ni, Ru, Rh, Pd, Ir, Pt, Au.

It is shown that Pd has the ability to absorb protons in its volume when it is in a negative potential compared to the potential of the electrolyte and becomes Palladium hydride (PdH). The onset reaction potential is pH dependent. The lower is the pH, the lower is the potential difference between Pd and source electrolyte. Proton transfer into Pd when the Pd is in lower potential between source and target at least −0.4 V vs Ag/AgCl in acidic solutions. In this potential difference proton transfer into the Pd, thus no accumulation occurs inside the membrane. However, the biomolecule, e.g. GABA, since it does not transfer into Pd, accumulates at the membrane and by drift-diffusion transfers in the target electrolyte. Hence, Pd selectively traps protons present in the electrolyte within the ion conductive channel by the formation of palladium hydride PdHₓ. It is expected that each of the other late transition metals will exhibit similar properties in this regard.

Metal hydrides and compounds with similar chemical properties may be selected from the group consisting of Aluminium hydride, Arsine, Beryllium hydride, Beryllium monohydride, Bismuthine, Borderline hydrides, Cadmium hydride, Caesium hydride, Calcium hydride, Calcium monohydride, Chlorobis(dppe)iron hydride, Chromium(II) hydride, Cobalt tetracarbonyl hydride, Complex metal hydride, Copper hydride, Digallane, Digermane, Diisobutylaluminium hydride, Germane, Hydrostannylation, Indium trihydride, Iron hydride, Iron tetracarbonyl hydride, Iron-hydrogen alloy, Iron(I) hydride, Iron(II) hydride, Knölker complex, Lithium aluminium hydride, Lithium hydride, Magnesium hydride, Magnesium iron hexahydride, Magnesium monohydride, Magnesium nickel hydride, Mercury(I) hydride, Mercury(II) hydride, Metal carbonyl hydride, Molybdocene dihydride, Mukaiyama hydration, Nickel hydride, Nickel-metal hydride, Palladium hydride, Pentacarbonylhydridomanganese, Pentacarbonylhydridorhenium, Plumbane, Plutonium hydride, Potassium hydride, Potassium nonahydridorhenate, Rubidium hydride, Scandium hydride, Scandium(III) hydride, Schwartz's reagent, Sodium aluminium hydride, Sodium bis(2-methoxyethoxy)aluminium hydride, Sodium hydride, Stannane, Stibine, Stryker's reagent, Thallium hydride, Titanium hydride, Titanium(IV) hydride, Transition metal hydride, Tributyltin hydride, Triphenyltin hydride, Uranium hydride, Uranium(IV) hydride, Yttrium hydride, Zinc hydride, Zirconium hydride, and Zirconium(II) hydride.

The term electrically conductive polymers refers to a wide group of polymer suitable for the application but they may be selected from the group consisting of polyanilines, which are described in Zhang, Z., Kashiwagi, H., Kimura, S. et al. A protonic biotransducer controlling mitochondrial ATP synthesis. Sci Rep 8, 10423 (2018), and polythiophenes, polypyrroles, polyisothianaphthalenes, polyphenylene vinylenes and copolymers thereof such as described by J C Gustafsson et al. in Solid State Ionics, 69, 145-152 (1994); Handbook of Oligo-and Polythiophenes, Ch 10.8, Ed D Fichou, Wiley-VCH, Weinhem (1999); by P Schottland et al. in Macromolecules, 33, 7051-7061 (2000); Technology Map Conductive Polymers, SRI Consulting (1999); by M Onoda in Journal of the Electrochemical Society, 141, 338-341 (1994); by M Chandrasekar in Conducting Polymers, Fundamentals and Applications, a Practical Approach, Kluwer Academic Publishers, Boston (1999); and by A J Epstein et al. in Macromol Chem, Macromol Symp, 51, 217-234 (1991). The electrically conductive polymer may be a polymer or copolymer of a 3,4-dialkoxythiophene, in which said two alkoxy groups may be the same or different or together represent an optionally substituted oxy-alkylene-oxy bridge. It is possible that the polymer is a polymer or copolymer of a 3,4-dialkoxythiophene selected from the group consisting of poly(3,4-methylenedioxythiophene), poly(3,4-methylenedioxythiophene) derivatives, poly(3,4-ethylenedioxythiophene), poly(3,4-ethylenedioxythiophene) derivatives, poly(3,4-propylenedioxythiophene), poly(3,4-propylenedioxythiophene) derivatives, poly(3,4-butylenedioxythiophene), poly(3,4-butylenedi-oxythiophene) derivatives, and copolymers therewith.

The electrically conductive polymer may be poly(3,4-ethylenedioxythiophene) (PEDOT). The electrodes may further comprise a polyelectrolyte compound, and said polyelectrolyte compound may be poly(styrene sulfonic acid) or a salt thereof. One possible material for use in the electrodes is poly(3,4-ethylenedioxythiophene) with a poly(styrene sulfonate) polyanion (in the following referred to as PEDOT:PSS). The electrodes may be present in the form of a thin layer of PEDOT:PSS deposited on a solid substrate.

The Bronsted base, especially Pd, may be present in or on the trapping electrode through alloying, electrodeposition, chemical vapor deposition, metal organic chemical vapor deposition, metal evaporation, sputter deposition, photocatalytic deposition, photochemical deposition, electroless plating, spray pyrolysis, thermal evaporation, cluster beam deposition, deposition via precipitation, gas-phase cluster deposition, chemical fluid deposition or other similar methods.

The minimum active amount of the Bronsted base is dependent on the ion to be delivered, the pH, and the Bronsted base used. In the case of GABA, as ion to be transported, and Pd, as active Bro/nsted base, optimal minimum concentration of Pd is twice the concentration of GABA.

The trapping electrode 300 is in ionic contact with the ion-conductive channel 302. The trapping electrode 300 may be in electric contact with the source electrode 200 and/or the target electrode 400.

The trapping electrode 300 may be positioned closer to the target electrolyte retainer 404 than to the source electrolyte retainer 204. The phrase ‘closer to the target electrolyte retainer’ refers to the trapping electrode 300 being placed in the half of the ion-conductive channel 302 that is closer to the target electrode 400. The position of the trapping electrode is may be closer to the outlet of the channel as it leads to an increase (negative value) for Iₜ which corresponds to a faster delivery of a higher concentration of ions.

The trapping electrode 300 may be positioned in any way that allows it to be in ionic contact with the ion-conductive channel 302. For example, the trapping electrode 300 may be placed on the support 102 and is embedded in the encapsulation layer 304 of the ion-conductive channel 302.

To supply and control a driving force for the electrophoretic delivery and to record the number of ions delivered and/or protons trapped the controller is configured to apply voltages between the different electrodes and record the current over time. The controller is further configured to change the voltages between the different electrodes independently and in various patterns to allow for an adjustment of delivered ions, and recovering of the trapping electrode, as well as the filling of the optional waste channel.

To drive the electrophoretic delivery of ions two voltages may be applied as input. A first voltage, Vₛ, between the source 200 and the trapping electrode 300 and a second voltage, Vₜ, between the target 400 and the trapping electrode 300. The second voltage, Vₜ, being smaller than the first voltage, Vₛ. The first voltage, Vₛ, can range from 0 to 240V and may be 1V and the second voltage, Vₜ, can range from 0 to 240V and may be 0.1-0.5V. Operating voltages for Vₛ, Vₜ, and V_{w} depend on dimensions and geometry of ion-conductive channel 302. The voltages used for medical devices may be less than 60V DC. The trapping electrode may be used as ground electrode.

The corresponding currents Iₛ and Iₜ may be recorded if both voltages, Vₛ, Vₜ, are simultaneously applied. Iₛ, which is the current between source and trapping electrode, may have a positive non-capacitive value of several μA. Due to its non-capacitive character Iₛ indicates transfer of protons in the trapping electrode and in the case of a Pd based trapping electrode it indicates the formation of PdHₓ. The recorded currents may be used to calculate the amount of ion delivered to the target, thereby allowing a high degree of precision in the drug delivered.

The controller may further be adapted to remove and/or reverse the potential of the trapping electrode 300 to allow the release of previously trapped protons into the ion-conductive channel 302 and hence recover the trapping electrode. The controller may also be adapted to remove and/or reverse the voltage Vₛ to facilitate the migration of protons towards the source electrode 300 within the source electrolyte 202.

The device 100 illustrated in FIG. 2 further comprises a waste channel 504 containing a waste electrolyte 502 and a waste electrode 500. The waste channel 504 is in ionic contact with the ion-conductive channel 302. The waste electrolyte 502 serves as a waste for the ions to be transported from the source electrolyte 202, and thus those ions may thus be soluble in the waste electrolyte 502. The waste channel 504 may be at least partially formed of a charge selective membrane, e.g. a CEM. The waste channel 504 may extend further into a part that does not contain a charge selective membrane and/or the waste channel 504 may extend into a waste electrolyte retainer which may or may not comprise a charge selective membrane.

The function of the waste electrode for a general OEIP is described in Tybrandt, K., Larsson, K. C., Kurup, S. et al. Translating Electronic Currents to Precise Acetylcholine -Induced Neuronal Signaling Using an Organic Electrophoretic Delivery Device. Adv. Mater. 21, 4442 (2009).

The waste electrode 500 may be in electric contact with any of the other electrodes. The waste electrode may be electrically connected with the source electrode 200. The controller may be adapted to apply a voltage, V_{w}, to the waste electrode 500. The waste electrode 500 may comprise a material or a combination of materials which is capable of electron-to-ion conversion, i.e. they need to enable charge transfer between the electrode and its contact and the electrodes may be so called non-polarizable electrodes. The materials used as waste electrode 500 are electrochemically active.

The waste may be used to carry away ions and to provide the means of a constant flow of ions through the ion-conductive channel 302. This facilitates the filling of the ion-conductive channel 302. By filling the waste channel 502 with mainly ions to be delivered to the target instead of a mix of protons and ions to be delivered, the amount of ions to be delivered stored within the device prior to release to the target is increased and hence a higher concentration of ions in the target can be achieved within a shorter timeframe.

The waste channel 504 can be oriented in any direction with respect to the ion-conductive channel 302. For example, the waste channel 504 may be in place with the ion-conductive channel 302 and is directly or indirectly attached to the support 102.

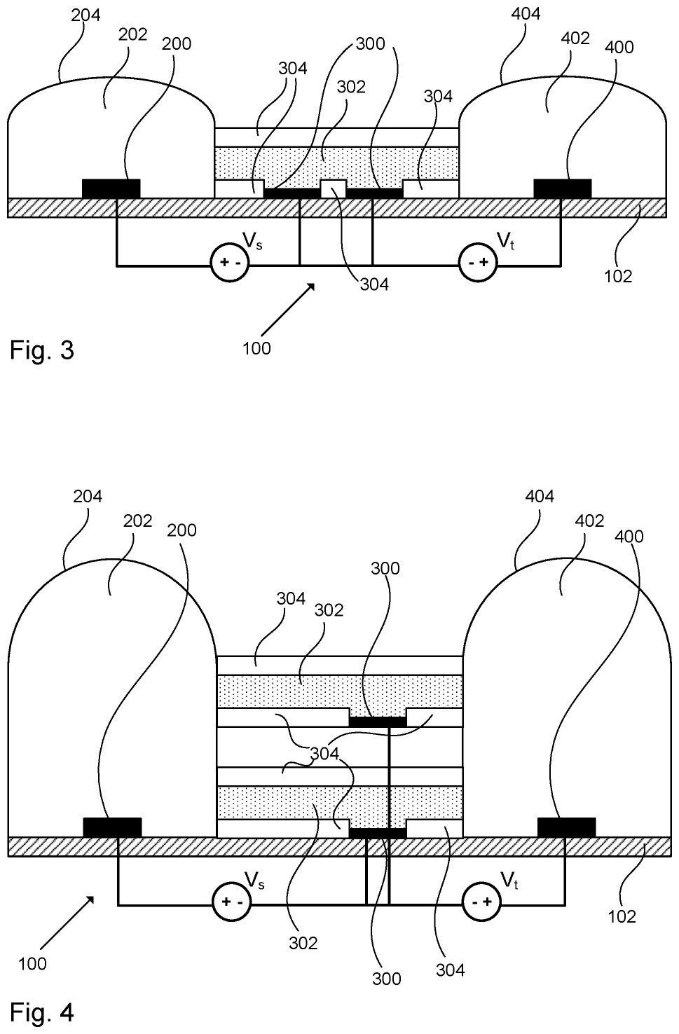

FIG. 3 illustrates a device as described previously for FIG. 1 with the addition of at least one second trapping electrode 300. The second trapping electrode 300 may be positioned in any way that allows it to be in ionic contact with the ion-conductive channel 302. The trapping electrode 300 may preferably be placed on the support 102 and may be embedded in the encapsulation layer 304 of the ion-conductive channel 302. The device 100 may comprise multiple trapping electrodes in at least one ion-conductive channel 302 having a length of 100 μm along the ion-conductive channel 302 and a spacing of 50 μm between the single trapping electrodes.

The second trapping electrode 300 may be comprised of the material of the first trapping electrode 300. The second trapping electrode 300 may be in electric contact with the source and/or target electrodes 200, 400. The second trapping electrode may be electrically connected in a fashion like the first trapping electrode 300.

As an alternative, the device 100 may comprise at least two ion conductive channels 302 connecting the source electrolyte 202 with the target electrolyte 402. Further, each of the ion-conductive channels 302 comprises at least one trapping electrode 300, which is in ionic contact with one ion-conductive channel 302 and which may be electrically connected to any of the other electrodes. Each of the ion-conductive channels 302 may be surrounded by an encapsulation layer 304.

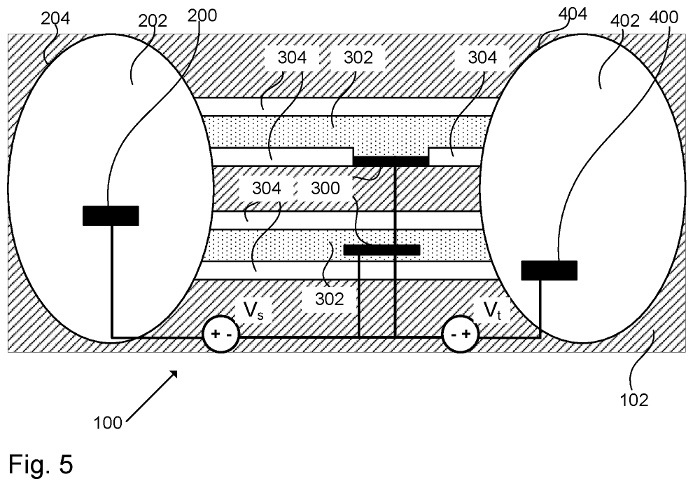

The at least two different ion-conductive channels 302 may be positioned in any way relative to each other, e.g. on top of each other as in FIG. 4 or next to each other in FIG. 5., if they are arranged to directly or indirectly connect the source electrolyte 202 with the target electrolyte 402.

The controller may be configured to independently apply potentials to the different trapping electrodes 300 to enable and/or enhance the selective transportation in one of the ion-conductive channels 302.

### REFERENCE LIST

100 device
102 support, electronically and ionically insulating
200 source electrode
202 source electrolyte
204 source electrolyte retainer
300 trapping electrode
302 ion-conductive channel
304 encapsulation layer
400 target electrode
402 target electrolyte
404 target electrolyte retainer
500 waste electrode
502 waste electrolyte
504 waste channel
Vₛ first voltage, between source and trapping electrode
Vₜ second voltage, between target and trapping electrode
V_{w} third voltage, between waste and trapping electrode

1. A device for electrophoretic delivery of ions comprising:
a source electrode electric and ionic contact with a source electrolyte, and
a target electrode in electric and ionic contact target electrolyte,
said source and target electrodes capable of conducting ions and electrons;
an ion-conductive channel connecting the source electrolyte with the target electrolyte to provide an ionic connection between said source and said target electrodes,
wherein said electrodes and said ion-conductive channel are formed of solid or semi-solid materials, and
a controller, operable to apply a drive voltage between said source and said target electrodes, such that at least after a voltage is applied across said ion-conductive channel, a potential difference between said source and target electrodes is provided,
the device further comprises a trapping electrode comprising an effective amount of a Bronsted base, said trapping electrode being arranged in ionic contact with the ion-conductive channel.

2. The device as claimed in claim 1, wherein the trapping electrode acts as a Bronsted acid after it was first loaded with protons and it is in a neural or positive potential versus the electrolyte potential.

3. The device as claimed in claim 1, wherein the trapping electrode is arranged in electric contact with at least one of the source and target electrodes.

4. The device as claimed in claim 1, wherein the ion-conductive channel is at least partially formed of a cation exchange membrane (CEM).

5. The device as claimed in claim 1, wherein the source electrolyte comprises a neurotransmitter, e.g. γ-aminobutyric acid (GABA) and/or glutamate, in a form suitable for transport by said cation exchange membrane.

6. The device as claimed in claim 1, wherein said controller is operable to apply a negative bias with respect to the other electrodes to the trapping electrode.

7. The device as claimed in claim 1, wherein said Bronsted base comprises at least one of the materials selected from the group consisting of late transition metals, metal hydrides, electrically conductive polymers and/or combinations thereof.

8. The device as claimed in claim 1, wherein the trapping electrode comprises at least an effective amount of the materials selected from the group consisting of late transition metals, metal hydrides, electrically conductive polymers and combinations thereof.

9. The device as claimed in claim 1, further comprising:
a source electrolyte retainer, for retaining the source electrolyte in contact with the source electrode, and/or
a target electrolyte retainer, for retaining the target electrolyte in contact with the target electrode.

10. The device as claimed in claim 1, wherein said trapping electrode is positioned closer to the target electrolyte retainer than to the source electrolyte retainer.

11-12. (canceled)

13. The device as claimed in claim 1, further comprising at least one waste channel, each waste channel comprising a waste electrolyte and a waste electrode.

14-22. (canceled)

23. A method of electrophoretically delivering ions from a source electrolyte to a target electrolyte comprising:
providing said ions in the source electrolyte,
providing an ion-conductive channel comprising a solid or semi-solid material, for ionically connecting the source electrolyte with the target electrolyte,
applying a first potential difference to a source electrode contacting the source electrolyte and a second potential to a target electrode contacting the target electrolyte, a difference between said first and second potentials being sufficient to drive said ions through the ion-conductive channel from the source electrolyte to the target electrolyte,
applying a third potential to a trapping electrode comprising a Bro/nsted base, wherein said third potential is sufficiently low so as to attract protons present in the ion-conductive channel, such that said protons are prevented from reaching the target electrolyte.

24. The method as claimed in claim 23, wherein the first potential is greater than the second potential and wherein the second potential is greater than the third potential.

25. (canceled)

26. The method as claimed in claim 23, wherein the first and second potentials are positive, and the third potential is negative.

27. The method as claimed in claim 26, wherein the third potential is lower than a lower potential of the first and the second potential such that protons are attracted to the trapping electrode while overall current is maintained to flow between the source electrolyte and target electrolyte.

28. The method as claimed in claim 23, further comprising recording a current applied to the target electrolyte as a function of time and calculating an amount of ions/drug/biomolecules delivered to the target electrolyte based on said recorded current.

29. The method as claimed in claim 23, further comprising releasing the third potential, whereby protons trapped at the trapping electrode are released.

30. The method as claimed in claim 23, further comprising a fourth potential being applied to the waste electrode to fill the waste channel with ions from the ion-conductive channel.

31. The device as claimed in claim 1, wherein the controller is configured to:
apply a first potential to the source electrode,
apply a second potential to the target electrode, said second potential being lower than the first potential, whereby cations are driven to migrate through the ion-conductive channel from the source electrolyte to the target electrolyte, and
apply a third potential to the trapping electrode, said third potential being lower than the second potential, whereby protons present in the ion-conductive channel are attracted to the trapping electrode.

32-33. (canceled)

34. A device for electrophoretically delivering ions from a source electrolyte to a target electrolyte,
wherein ions are provided in the source electrolyte, the device comprising:
an ion-conductive channel comprising a solid or semi-solid material, for ionically connecting the source electrolyte with the target electrolyte,
a controller configured to:
apply a first potential difference to a source electrode contacting the source electrolyte and a second potential to a target electrode contacting the target electrolyte, a difference between said first and second potentials being sufficient to drive said ions through the ion-conductive channel from the source electrolyte to the target electrolyte, and
apply a third potential to a trapping electrode comprising a Bronsted base, wherein said third potential is sufficiently low so as to attract protons present in the ion-conductive channel, such that said protons are prevented from reaching the target electrolyte.

35-41. (canceled)
